(19) United States
(12) Patent Application Publication
(10) Pub. No.: US 2008/0038248 A1
(43) Pub. Date: Feb. 14, 2008

(54) Proteins Ligands For Nkg2d And Ul16 Receptors And Uses Thereof

(76) Inventors: John Trowsdale, Cambridgeshire (GB); Robert Eagle, Cambridgeshire (GB); Louise Bacon, Cambridgeshire (GB)

Correspondence Address: COOLEY GODWARD KRONISH LLP;ATTN: Patent Group, Suite 1100, 777 - 6th Street, NW, WASHINGTON, DC, 20001, US

(21) Appl. No.: 10/589,851

(22) PCT Filed: Feb. 17, 2005
(86) PCT No.: PCT/GB05/00572

§ 371 Date: Jun. 4, 2007

(30) Foreign Application Priority Data
Feb. 17, 2004 (GB) 0403491.4

(51) Int. Cl. A61K 39/395; A61K 31/711; A61P 35/00; C07H 21/04; C07K 16/00; C12N 15/63; G01N 33/53; G01N 33/50; C12N 1/21; C07K 14/00; A61P 37/00; A61K 38/16

(52) U.S. Cl. 424/130.1; 435/252.3; 435/320.1; 435/69.1; 435/7.8; 436/86; 514/12; 514/44; 530/350; 530/387.9; 536/23.1

(57) ABSTRACT

A novel member of the RAET1/ULBP family of proteins (RAET1G) is identified and characterised and shown to bind the UL16 and NKG2D receptors with high affinity. RAET1G exhibits restricted expression in normal tissues but exhibits high levels of expression in tumours and may be differentially spliced in cancer cells to produce a soluble protein. RAET1G is also up-regulated in inflammatory diseases such as coeliac disease. RAET1G may be used as a novel marker for disease conditions including cancer conditions and inflammatory diseases.

This invention relates to protein ligands which interact with the cell surface receptor NKG2D and the viral ligand UL16.

The C-type lectin-like receptor NKG2D has a number of defined, MHC class I-related ligands in both mouse and man. Murine ligands include the retinoic acid early transcript 1 (Rae-1) family, the minor histocompatibilty antigen H60, and the recently identified MULT1 (Cerwenka et al., (2000) Immunity 12, 721-727; Diefenbach et al., (2000) Nat. Immunol. 1, 119-126; Carayannopoulos et al. (2002), J. Immunol. 169, 4079-4083). Human ligands include the MHC class I chain-related genes MICA and MICB (Bauer et al. (1999) Science 285, 727-729) and the UL16-binding protein ULBP family (Cosman et al. (2001) Immunity 14, 123-133).

MIC proteins have three α domains structurally similar to those of classical MHC class I molecules, but they do not bind peptides or associate with β2 microglobulin. H60, ULBP1-3 and the Rae-1 family only possess MHC-like α1α2 domains. The human ULBP and murine Rae-1 proteins are distinct from the other NKG2D ligands, as they are GPI anchored to the membrane, rather than possessing a transmembrane (TM) region.

NKG2D exists as a homodimer at the cell surface. Diverse ligands bind to only five conserved ‘hotspots’ within the NKG2D binding site (McFarland et al. Immunity (2003) 19, 803-812; McFarland et al Structure (2003) 11, 411-422). NKG2D is not limited to NK cells and is also expressed on activated CD8⁺ T cells, γδ T cells, and activated macrophages (Jamieson et al. (2002) Immunity 17, 19-29).

The expression of NKG2D ligands is poorly understood.

MIC is frequently expressed on tumours of epithelial origin (Groh et al. (1999) Proc. Natl. Acad. Sci. USA 96, 6879-6884) and up-regulation of NKG2D ligands on tumours may be a mechanism for immune recognition and elimination of malignant cells. A study of tumor susceptibility to NKG2D-dependent natural killer cell cytotoxicity indicates that the involvement of NKG2D in natural killer cell-mediated cytotoxicity strictly correlates with the expression and the surface density of MICA and ULBP on target cell tumors of different histotypes (Pende et al. (2002) Cancer Res., 62, 6178-6186). In mouse models, implanted tumour cells transfected with NKG2D ligands invoked potent antitumour immunity and rejection of tumour cells in vivo (Diefenbach et al. (2000) Nat. Immunol. 1, 119-126; Cerwenka et al. (2001) Proc. Natl. Acad. Sci. USA 98, 11521-11526; Diefenbach et al. (2001) Nature 413, 165-171; Girardi et al. (2001) Science 294, 605-609). NKG2D ligands may also have a role in the immune response to pathogens, including cytomegalovirus (Groh et al. (2001) Immunol. 2, 255-260) Mycobacterium tuberculosis (Das et al. (2001) Immunity 15, 83-93) and Escherichia coli (Tieng et al. (2002) Proc. Natl. Acac. Sci. 99, 2977-2982).

NK cell function is impaired in non-obese diabetic (NOD) mice by expression of NKG2D ligands on the NK cells (Ogasawara et al., (2003) Immunity 18, 41-51). In rheumatoid arthritis, interaction of the NKG2D receptor with its ligands is impaired (Groh et al., (2003) Proc. Natl. Acad. Sci. USA 100, 9452-9457).

Expression of Unique Long (UL) 16 glycoprotein by human cytomegalovirus (hCMV) may be a mechanism by which hCMV evades immune recognition by interfering with NKG2D binding to its ligands (Cosman et al. (2001) Immunity 14, 123-133; Welte et al. (2003) Eur. J. Immunol. 33, 194-203). Not all human MIC and ULBP proteins are targeted. MICB, ULBP1, and ULBP2 are bound by UL16 whereas MICA and ULBP3 are not. Similarly, different murine ligands have variable affinities for NKG2D (Carayannopoulos et al. (2002) J. Immunol. 169, 4079-4083; O'Callaghan et al. (2001) Immunity 15, 201-211; Carayannopoulos et al (2002) Eur. J. Immunol. 32, 597-605). MIC and ULBP proteins can be expressed independently of each other on cells of different lineages, which is also consistent with non-redundant functions (Pende et al. (2002) Cancer Res. 62, 6178-6186)

A number of ULBP-related genes (the ‘RAE1-like transcripts’ (RAET1)) have been identified in a cluster on chromosome 6p24.2-q25.3 (Radosavljevic et al. (2002) Genomics 79, 114-123). This cluster includes several new genes distinct from ULBP1-3, including RAET1E (US2003/0195337).

The present invention relates to the identification and characterisation of a novel member of the RAET1/ULBP family of proteins, termed ‘RAET1G’. RAET1G is shown herein to bind the UL16 and NKG2D receptors with an affinity significantly higher than any of the ULBP family of proteins reported to date.

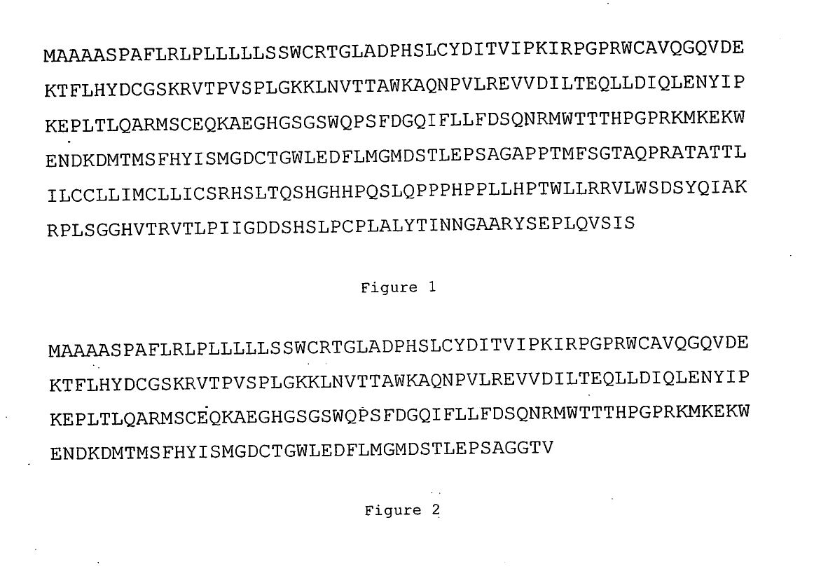

One aspect of the present invention provides an isolated nucleic acid encoding a polypeptide which comprises or consists of an amino acid sequence having at least 87% sequence identity or at least 87% sequence similarity with the amino acid sequence of FIG. 1 or FIG. 2.

The amino acid sequence of FIG. 1 (RAETG1) has the database number AAO22238.1, GI:37728026 and is encoded by the sequence of database number AY172579.1, GI:37728025.

The amino acid sequence of FIG. 2 (RAETG2) has the database number AAO22239.1 GI:37728028 and is encoded by the sequence of database number AY172580.1, GI:37728027.

The polypeptide may comprise or consist of an amino acid sequence of at least 90% sequence identity or similarity, at least 95% sequence identity or similarity, or at least 98% sequence identity or similarity with the amino acid sequence of FIG. 1. In some preferred embodiments, the polypeptide may comprise or consist of the amino acid sequence of FIG. 1 and/or FIG. 2.

Preferably, the polypeptide has one or more RAET1G functions. For example, the polypeptide may bind to UL16 (coding sequence AY297445, AY297445.1, GI:31616608; protein sequence AAP55721; AAP55721.1; GI:31616609) and/or NKG2D (coding seq AF461811, AF461811.1, GI:18182679; protein sequence AAL65233, AAL65233.1, GI:18182680), preferably with high affinity (i.e. 360 nM or less)

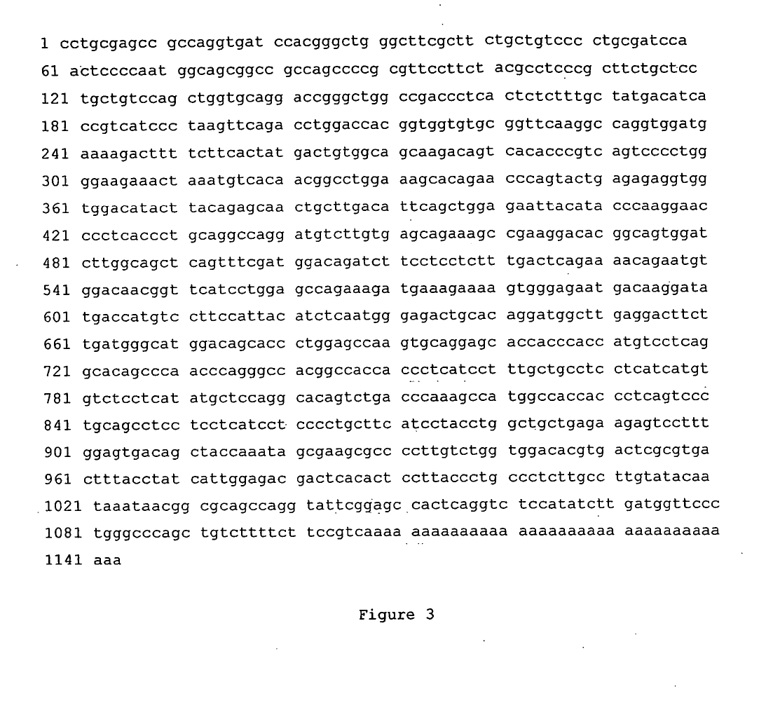

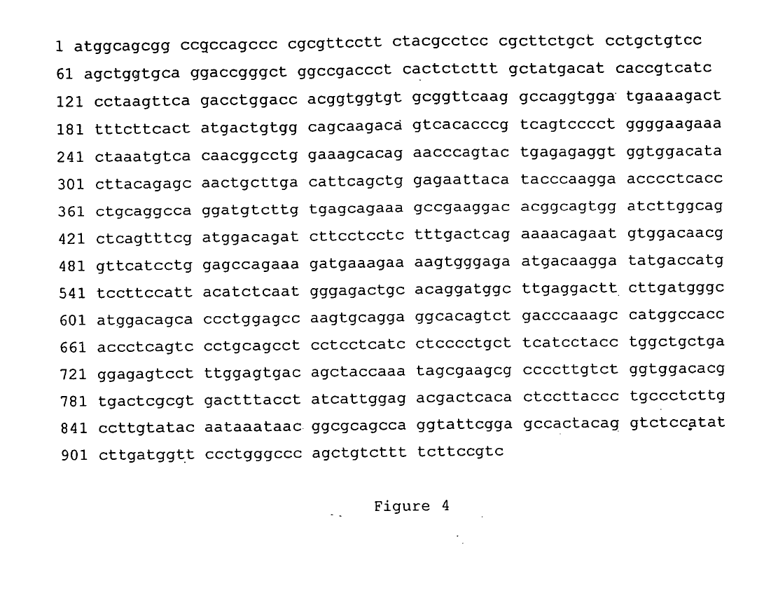

An isolated nucleic acid as described herein may share greater than about 85% sequence identity with the nucleic acid sequence of FIG. 3 or FIG. 4, greater than about 90%, or greater than about 95%.

The nucleic acid may comprise or consist of a sequence shown in FIG. 3 or FIG. 4, it may be a mutant, variant, derivative or allele of the sequence shown. The sequence may differ from that shown by a change that is one or more of addition, insertion, deletion and substitution of one or more nucleotides of the sequence shown. Changes to a nucleic acid sequence may result in an amino acid change at the protein level, or not, as determined by the genetic code.

Thus, a nucleic acid may include a sequence different from the sequence shown in FIG. 3 or FIG. 4, yet encode a polypeptide with the same amino acid sequence.

Sequence identity is described in more detail below.

A nucleic acid of the invention may hybridise with the sequence shown in FIG. 3 and/or FIG. 4 under stringent conditions, or may have a complement which hybridises to the sequence shown in FIG. 3 and/or FIG. 4 under stringent conditions. Suitable conditions include, e.g. for sequences that are about 80-90% identical, suitable conditions include hybridisation overnight at 42° C. in 0.25M Na₂HPO₄, pH 7.2, 6.5% SDS, 10% dextran sulphate and a final wash at 55° C. in 0.1×SSC, 0.1% SDS. For sequences that are greater than about 90% identical, suitable conditions include hybridisation overnight at 65° C. in 0.25M Na₂HPO₄, pH 7.2, 6.5% SDS, 10% dextran sulphate and a final wash at 60° C. in 0.1×SSC, 0.1% SDS. Preferably, a nucleic acid encodes a polypeptide with RAET1G activity, as described above.

The invention also includes fragments of nucleic acid sequences as described herein, for example, a fragment of the nucleotide sequence of FIG. 3 or FIG. 4. Suitable fragments may consist of less than 891 nucleotides, for example from 10, 20, 30, 40 or 50 nucleotides to 800, 870, 880 or 891 nucleotides. Such a fragment may encode a RAET1G polypeptide as described below, or may be useful as an oligonucleotide probe or primer.

Another aspect of the present invention provides an isolated RAET1G polypeptide encoded by a nucleic acid sequence described above, for example the nucleic acid sequence of FIG. 3 or 4.

A polypeptide may comprise or consist of the amino acid sequence shown in FIG. 1 and/or FIG. 2 or may be a variant, allele, derivative or mutant thereof.

A variant, allele, derivative or mutant of an RAET1G polypeptide as described herein may include a polypeptide modified by varying the amino acid sequence of the protein, e.g. by manipulation of the nucleic acid encoding the protein or by altering the protein itself. Such derivatives of the natural amino acid sequence may involve one or more of insertion, addition, deletion or substitution of one or more amino acids, which may be without fundamentally altering the qualitative activity of the polypeptide, for example the binding of the polypeptide to the UL16 receptor and/or the NKG2D receptor.

A variant, allele, derivative or mutant may comprise an amino acid sequence which shares greater than about 87% sequence identity with the sequence of FIG. 1, greater than about 90% or greater than about 95%. The sequence may share greater than about 87% similarity with the amino acid sequence of FIG. 1 and/or FIG. 2, or greater than about 90% similarity. Preferably, an amino acid sequence variant, allele, derivative or mutant of an RAET1G polypeptide retains binding affinity for the UL16 receptor and/or the NKG2D receptor.

Sequence similarity and identity are commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4.

Use of GAP may be preferred but other algorithms may be used, e.g. BLAST (which uses the method of Altschul et al. (1990) J. Mol. Biol. 215, 405-410), FASTA (which uses the method of Pearson and Lipman (1988) Proc. Natl. Acad. USA 85, 2444-2448), or the Smith-Waterman algorithm (Smith and Waterman (1981) J. Mol. Biol. 147, 195-197), or the TBLASTN program, of Altschul et al. (1990) supra, generally employing default parameters. In particular, the psi-Blast algorithm (Nucl. Acids Res. (1997) 25, 3389-3402) may be used. Sequence identity and similarity may also be determined using Genomequest™ software (Gene-IT, Worcester Mass. USA).

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Particular amino acid sequence variants may differ from a known polypeptide sequence as described herein by insertion, addition, substitution or deletion of 1 amino acid, 2, 3, 4, 5-10, 10-20 20-30, 30-50, or more than 50 amino acids.

Sequence comparison is made over the full-length of the relevant sequence described herein, except when context dictates otherwise.

An RAET1G polypeptide may include a polypeptide fragment which consists of fewer amino acid residues than the full-length polypeptide, for example the full length sequence of FIG. 1 and/or FIG. 2. Such a fragment may consist of at least 110 amino acids, more preferably at least 160 amino acids, more preferably at least 200 amino acids, more preferably at least 250 amino acids, more preferably at least 297 amino acids. Such a fragment may consist of 297 amino acids or less, 250 amino acids or less, or 160 amino acids or less, or 110 amino acids or less.

Preferably, a polypeptide as described herein comprises α1 and α2 domains corresponding to at least residues 83 to 202 in the sequence of FIG. 1 or FIG. 2.

The polypeptide may also comprise a transmembrane domain corresponding to at least residues 227-242 in the sequence of FIG. 1 or FIG. 2, and/or a cytoplasmic domain corresponding to at least residues 243-297 or fewer in the sequence of FIG. 1 or FIG. 2.

A polypeptide as described herein may further comprise a proline residue at a position corresponding to position 163 of the amino acid sequence of FIG. 1 or FIG. 2.

Amino acid residues are described in the present application with reference to their position in the sequence of FIG. 1. It will be appreciated that the equivalent residues in other RAET1G polypeptides may have a different position and number, because of differences in the amino acid sequence of each polypeptide. These differences may occur, for example, through variations in the length of the N terminal domain. Equivalent residues in RAET1G polypeptides are easily recognisable by their overall sequence context and by their positions with respect to the α1 and α2 domains.

A polypeptide as described herein may be soluble or insoluble, for example a polypeptide may be anchored to or within a membrane.

Preferably, a polypeptide has RAET1G function and binds with high affinity to a UL16 receptor and/or a NKG2D receptor. The affinity of an RAET1G polypeptide for a UL16 and/or an NKG2D receptor may be determined by any one of a range of standard techniques, including for example, surface plasmon resonance.

High affinity binding to a receptor is, in general, binding of sub-micromolar affinity. Moderate-low affinity binding is, in general, binding of micromolar or tens of micromolar affinity.

RAET1G polypeptides as described herein bind with a comparable or, more preferably, a higher affinity than the binding affinity of other NKG2D ligands, such as ULBP1 with an affinity for NKG2D of 1.68 μM.

An RAET1G polypeptide may also comprise additional amino acid residues which are heterologous to the RAET1G sequence. For example, an RAET1G polypeptide as described above may be included as part of a fusion protein, where the heterologous amino acid residues enable the fusion protein to have a function in addition to binding affinity for the UL16 and/or NKG2D receptors. For example, the additional function may provide a desired property, or may allow an agent with a desired property to be joined to the fusion protein.

In some embodiments, a RAET1G polypeptide may be chemically attached to a functional moiety in a conjugate. Functional moieties which may be conjugated with a RAET1G polypeptide include polypeptides, non-peptidyl chemical compounds, cells and virus particles. A functional moiety may, for example, have cytotoxic activity or a binding activity.

The skilled person can use the techniques described herein and others well known in the art to produce large amounts of polypeptides and peptides, for instance by expression from encoding nucleic acid.

A method of producing an RAET1G polypeptide may comprise;

(a) causing expression from a nucleic acid which encodes a RAET1G polypeptide to produce the RAET1G polypeptide recombinantly; and,

(b) testing the recombinantly produced polypeptide for RAET1G activity.

Suitable nucleic acid sequences include a nucleic acid sequence encoding an RAET1G polypeptide as described above.

A polypeptide may be isolated and/or purified (e.g. using an antibody for instance) after production by expression from encoding nucleic acid (for which see below). Thus, a polypeptide may be provided free or substantially free from contaminants with which it is naturally associated (if it is a naturally-occurring polypeptide). A polypeptide may be provided free or substantially free of other polypeptides.

Fusion polypeptides may be generated to facilitate purification of the RAET1G polypeptide. For example, six histidine residues may be incorporated at either the N-terminus or C-terminus of the recombinant protein. Such a histidine tag may be used for purification of the protein by using commercially available columns which contain a metal ion, either nickel or cobalt (Clontech, Palo Alto, Calif., USA).

The recombinantly produced polypeptide may be isolated and/or tested for RAET1G activity by determination of the binding affinity for the UL16 receptor and/or the NKG2D receptor by incubation of the RAET1G polypeptide with the receptor and quantification of binding affinity using surface plasmon resonance.

An isolated nucleic acid as described above, for example a nucleic acid encoding an RAET1G polypeptide, may be comprised in a vector. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Further aspects of the present invention provide a host cell containing heterologous nucleic acid encoding an RAET1G polypeptide as described above.

Host cells, in particular host cells which are cancer cells, may be useful in the treatment of a cancer condition, for example by stimulating an immune response to the cancer cells in the host organism.

The nucleic acid may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

The introduction of nucleic acid into a host cell, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium.

Following production by expression, a polypeptide may be isolated and/or purified from the host cell and/or culture medium, as the case may be, tested for RAET1G activity and subsequently used as desired, e.g. in the formulation of a composition which may include one or more additional components, such as a pharmaceutical composition which includes one or more pharmaceutically acceptable excipients, vehicles or carriers (e.g. see below).

In other embodiments, the host cell comprising the expressed polypeptide, for example at the cell surface, may be isolated and/or purified and formulated in a pharmaceutical composition, for example for the treatment of a cancer or other RAET1G mediated condition.

Another aspect of the present invention provides an isolated antibody that binds specifically to a RAET1G polypeptide.

Antibodies may be obtained using techniques that are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al., (1992) Nature 357, 80-82). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed, the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Antibodies which specifically bind to a RAET1G polypeptide may, for example, be useful in determining whether an individual has a disease condition such as cancer.

It is shown herein that RAET1G exhibits restricted expression in normal tissues but exhibits high levels of expression in tumours, in particular tumours of epithelial origin. RAET1G may therefore be useful as a tumour cell marker in the diagnosis and grading of tumours.

The data presented herein shows that expression of RAET1G is increased in the small intestine in coeliac disease. RAET1G expression may also be increased in other inflammatory diseases of the gut, such as Crohn's disease.

A method of identifying a cancer condition or inflammatory disease in an individual may comprise:

determining the expression of a RAET1G polypeptide in a sample obtained from the individual.

Increased expression of RAET1G polypeptide in the test sample relative to controls may be indicative that the individual has the condition or disease.

Expression of a RAET1G polypeptide may be determined by determining the presence or amount of RAET1G polypeptide in the sample.

Cancer cells are shown herein to differentially splice RAET1G transcripts to produce a truncated RAET1G polypeptide which lacks the RAET1G transmembrane and cytoplasmic domains (i.e. a soluble RAET1G polypeptide).

A method of identifying a cancer condition in an individual may comprise:

determining the expression of a soluble RAET1G polypeptide in a sample obtained from the individual.

Increased expression of soluble RAET1G polypeptide in the test sample relative to controls may be indicative that the individual has the condition or disease.

Expression of a soluble RAET1G polypeptide may be determined by determining the presence or amount of soluble RAET1G polypeptide in the sample.

Suitable controls are well known to the skilled person and may include, for example, a sample obtained from a healthy individual. The sample obtained from a healthy individual may be taken from a different individual to that in which the condition is being identified, or it may be a sample taken from the same individual at a different time.

A cancer condition may, for example, include leukaemia conditions such as T-cell leukaemia, or epithelial cancer, which may include a cancer of the kidney, liver, lung, oesophagus, ovary (serous carcinoma), skin, endometrioid carcinoma of the uterus and/or squamous carcinoma of the uterus. An inflammatory disease may include coeliac disease or Crohn's disease.

The presence or amount of RAET1G polypeptide may be determined directly by contacting the sample with an antibody as described herein.

A soluble RAET1G polypeptide may comprise or consist of the amino acids 1-213 of the full length RAET1G sequence.

The sample may be a tissue biopsy sample, for example from tissue suspected of disease or malignancy, or may be a biological fluid sample, for example from blood, serum or plasma. A biological sample may comprise cells which may, optionally, be concentrated and/or isolated prior to contacting with the antibody.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be direct or indirect, covalent, e.g. via a peptide bond, or non-covalent. Linkage via a peptide bond may be as a result of recombinant expression of a gene fusion that encodes antibody and reporter molecule. The mode of determining binding is not a feature of the present invention and those skilled in the art are able to choose a suitable mode according to their preference and general knowledge.

For example, a range of conventional techniques are available to determine and/or quantify the binding of antibody to RAET1G polypeptide, including for example, HPLC or ELISA.

An antibody as described herein may be a component of a kit for detecting a cancer condition in an individual, using a method as described herein.

In other embodiments, the expression of a soluble RAET1G polypeptide may be determined indirectly by determining the level of nucleic acid encoding soluble RAET1G in the sample.

A method of identifying a cancer condition in an individual may comprise;
determining the presence or amount of nucleic acid encoding a soluble RAET1G polypeptide in a sample obtained from the individual.

Nucleic acid encoding a soluble RAET1G polypeptide may include a nucleic acid which encodes the amino acid sequence of FIG. 1 of the RAET1G sequence. A suitable nucleic acid may, for example, comprise or consist of the sequence of FIG. 3, or be an allele or variant thereof.

The presence or amount of a nucleic acid, in particular an RNA, may be determined by any convenient techniques, including, for example RT-PCR or Northern Blotting.

The invention also encompasses the use of an RAET1G polypeptide as described herein in a method for obtaining or identifying a modulator, for example an inhibitor, of RAET1G and/or its interaction with UL16 and/or NKG2D receptors.

A method for obtaining and/or identifying a modulator of a RAET1G polypeptide may comprise;

(a) bringing into contact a RAET1G polypeptide and a test compound; and,

(b) determining the interaction of the RAET1G polypeptide with the test compound.

In other embodiments, a method for obtaining and/or identifying a modulator of a RAET1G polypeptide may comprise;

(a) bringing into contact a RAET1G polypeptide and a UL16 or NKG2D polypeptide in the presence of a test compound; and,

(b) determining the interaction between the UL16 or NKG2D polypeptide and the RAET1G polypeptide.

Interaction or binding may be determined in the presence and absence of test compound. A difference in interaction or binding in the presence of the test compound relative to the absence of test compound may be indicative of the test compound being a modulator of RAET1G activity.

Polypeptides may be contacted under conditions wherein, in the absence of the test compound, the polypeptides interact or bind to each other. The RAET1G polypeptide may be in the reaction medium in an isolated form or may be comprised on a cell membrane.

Methods for obtaining or identifying RAET1G modulators as described herein may be in vivo cell-based assays, or in vitro non-cell-based assays. In in vitro assays, the RAET1G polypeptide may be isolated, fixed to a solid support or comprised on a membrane. Suitable cell types for in vivo assays include mammalian cells such as CHO, HeLa and COS cells.

The precise format of the methods described herein may be varied by those of skill in the art using routine skill and knowledge.

It is not necessary to use the entire full-length RAET1G, UL16 or NKG2D polypeptides for in vitro or in vivo assays of the invention. Polypeptide fragments as described herein which retain the activity of the full length protein may be generated and used in any suitable way known to those of skill in the art.

For example, binding affinity may be studied in vitro by immobilising either the RAET1G polypeptide or one or both of the UL16 and NKG2D receptor to a solid support, then bringing it into contact with the other. The binding affinity can then be determined by standard techniques, such as surface plasmon resonance. The RAET1G polypeptide or the receptor may be labelled with a detectable label. Suitable detectable labels include ³⁵S-methionine which may be incorporated into recombinantly produced peptides and polypeptides.

Recombinantly produced peptides and polypeptides may also be expressed as a fusion protein containing an epitope which can be labelled with an antibody.

A method described herein may be performed in vivo, for example in a cell line such as a yeast or mammalian cell line in which the relevant polypeptides or peptides are expressed from one or more vectors introduced into the cell.

The ability of a test compound to modulate interaction between a RAET1G polypeptide and a UL16 or NKG2D polypeptide may be determined using a so-called two-hybrid assay. For example, a polypeptide or peptide containing a fragment of a RAET1G polypeptide or UL16 or NKG2D polypeptide as the case may be, or a peptidyl analogue or variant thereof as disclosed, may be fused to a nucleic acid binding domain such as that of the yeast transcription factor GAL 4. The GAL 4 transcription factor includes two functional domains. These domains are the DNA binding domain (GAL4 DBD) and the GAL4 transcriptional activation domain (GAL4TAD). By fusing one polypeptide or peptide to one of those domains and another polypeptide or peptide to the respective counterpart, a functional GAL 4 transcription factor is restored only when two polypeptides or peptides of interest interact. Thus, interaction of the polypeptides or peptides may be measured by the use of a reporter gene probably linked to a GAL 4 DNA binding site that is capable of activating transcription of said reporter gene. This assay format is described by Fields and Song, 1989, Nature 340; 245-246. This type of assay format can be used in both mammalian cells and in yeast.

Other combinations of nucleic acid binding domain and transcriptional activation domain are available in the art and may be preferred, such as the LexA DNA binding domain and the VP60 transcriptional activation domain.

In some embodiments, the RAET1G, UL16 or NKG2D polypeptide or peptide may be employed as a fusion with (e.g.) the LexA DNA binding domain, and the counterpart (e.g.) UL16, NKG2D or RAET1G, polypeptide or peptide as a fusion with (e.g.) VP60, and involves a third expression cassette, which may be on a separate expression vector, from which a peptide or a library of peptides of diverse and/or random sequence may be expressed. A reduction in reporter gene expression (e.g. in the case of β-galactosidase a weakening of the blue colour) results from the presence of a peptide which disrupts the RAET1G/receptor (for example) interaction, which interaction is required for transcriptional activation of the β-galactosidase gene. Where a test substance is not peptidyl and may not be expressed from encoding nucleic acid within a said third expression cassette, a similar system may be employed with the test substance supplied exogenously.

When performing a two hybrid assay to look for substances which interfere with the interaction between two polypeptides or peptides it may be preferred to use mammalian cells instead of yeast cells. The same principles apply and appropriate methods are well known to those skilled in the art.

The RAET1G, UL16 and/or NKG2D polypeptides may be present on and/or in a cell or different cells. This may be achieved, for example by expressing the polypeptides from one or more expression vectors which have been introduced into the cell by transformation.

A suitable UL16 polypeptide may include Human Cytomegalovirus (HCMV) UL16 (Acc No. AY297445) or a variant, homologue, mutant, allele or derivative thereof. A variant, allele, derivative, homologue, or mutant of UL16 may consist of a sequence having greater than about 70% sequence identity with the sequence of HCMV UL16, greater than about 80%, greater than about 90%, or greater than about 95%.

A suitable NKG2D receptor may include the human NKG2D receptor (Acc No. AF481811) or a variant, homologue, mutant, allele or derivative thereof. A variant, allele, derivative, homologue, or mutant of NKG2D may consist of a sequence having greater than about 70% sequence identity with the sequence of human NK cell NKG2D receptor, greater than about 80%, greater than about 90%, or greater than about 95%.

The amount of test substance or compound which may be added to an assay of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM. When cell-based assays are employed, the test substance or compound is desirably membrane permeable in order to access the RAET1G polypeptide.

Test compounds may be natural or synthetic chemical compounds used in drug screening programmes. Extracts of plants which contain several characterised or

uncharacterised components may also be used.

Combinatorial library technology (Schultz, (1996) Biotechnol. Prog. 12, 729-743) provides an efficient way of testing a potentially vast number of different substances for ability to modulate activity of a polypeptide.

One class of test compounds can be derived from the RAET1G, UL16 and/or NKG2D polypeptides. Membrane permeable peptide fragments of from 5 to 40 amino acids, for example, from 6 to 10 amino acids may be tested for their ability to modulate such interaction or activity.

Peptides can also be generated wholly or partly by chemical synthesis according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.). Peptides may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulphonic acid or a reactive derivative thereof. The modulatory properties of a peptide may be enhanced by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond Oxford University Press 2001).

Other candidate modulator compounds may be based on modelling the 3-dimensional structure of a polypeptide or peptide fragment and using rational drug design to provide potential inhibitor compounds with particular molecular shape, size and charge characteristics. This is described in more detail below.

Antibodies directed to RAET1G polypeptide may form a further class of putative modulator compounds. Candidate antibodies may be characterised and their binding regions determined to provide single chain antibodies and fragments thereof which are responsible for modulating the interaction.

Following identification of a compound using a method described above, the compound may be isolated and/or synthesised.

An agent identified using one or more primary screens (e.g. in a cell-free system) as having ability to interact with RAET1G and/or a receptor, such as UL16 or NKG2D, and/or modulate activity of RAET1G may be assessed or investigated further using one or more secondary screens. Biological activity, for example, may be tested in an NK cell cytotoxicity assay. Test compounds found to modulate the activity of RAET1G may be tested for activity in inhibiting NK cell cytotoxicity.

Following identification of a compound as described above, a method may further comprise modifying the compound to optimise the pharmaceutical properties thereof.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the optimisation of the lead compound.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

The test compound may be manufactured and/or used in preparation, i.e. manufacture or formulation, of a composition such as a medicament, pharmaceutical composition or drug. These may be administered to individuals, e.g. for any of the purposes discussed elsewhere herein.

A method of the invention may comprise formulating said test compound in a pharmaceutical composition with a pharmaceutically acceptable excipient, vehicle or carrier as discussed further below.

Another aspect of the present invention provides a method of producing a pharmaceutical composition comprising;

i) identifying a compound which modulates the activity of an RAET1G polypeptide using a method described herein; and,

ii) admixing the compound identified thereby with a pharmaceutically acceptable carrier.

The formulation of compositions with pharmaceutically acceptable carriers is described further below.

Another aspect of the invention provides a method for preparing a pharmaceutical composition, for example, for the treatment of a condition which is mediated by RAET1G, comprising;

i) identifying a compound which is an agonist or antagonist of a RAET1G polypeptide

ii) synthesising the identified compound, and;

iii) incorporating the compound into a pharmaceutical composition.

The identified compound may be synthesised using conventional chemical synthesis methodologies. Methods for the development and optimisation of synthetic routes are well known to persons skilled in this field.

The compound may be modified and/or optimised as described above.

Incorporating the compound into a pharmaceutical composition may include admixing the synthesised compound with a pharmaceutically acceptable carrier or excipient.

Another aspect of the present invention provides a modulator, for example an inhibitor, of RAET1G activity, or composition comprising such a modulator, which is isolated and/or obtained by a method described herein.

Suitable modulators may include small chemical entities, peptide fragments, antibodies or mimetics as described above.

Another aspect of the invention provides a pharmaceutical composition comprising a modulator as described herein and a pharmaceutically acceptable excipient, vehicle or carrier.

Another aspect of the invention provides an RAET1G polypeptide or fragment thereof, or a nucleic acid encoding a RAET1G polypeptide or fragment thereof, or an antibody, cell or a modulator, as described above, for use in the treatment of a human or animal body.

Another aspect of the invention provides the use of a RAET1G polypeptide or fragment thereof, nucleic acid encoding a RAET1G polypeptide or fragment thereof, antibody as described herein, host cell as described herein, or a modulator obtained by a method described herein, in the manufacture of a composition for the treatment of an individual with a disorder mediated by RAET1G.

A disorder mediated by RAET1G may include a pathogenic infection, a cancer condition or an immune disorder.

A pathogenic infection may include a bacterial infection, such as an Mycobacterium tuberculosis or Escherichia coli infection or a viral infection, such as a human cytomegalovirus infection.

A cancer condition may include lung cancer, gastrointestinal cancer, bowel cancer, colon cancer, breast carcinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma, lymphoma or leukaemia.

A condition of the immune system may include autoimmune diseases, such as coeliac disease, rheumatoid arthritis, lupus, scleroderma, Sjögren's syndrome and multiple sclerosis, diabetes or inflammatory bowel diseases such as inflammatory bowel syndrome, ulcerative colitis and Crohn's disease. It may also be useful for patients who have undergone transplant surgery, to reduce or prevent rejection.

Whether it is a cell, polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil.

Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Aspects of the present invention will now be illustrated with reference to the accompanying figures described below and experimental exemplification, by way of example and not limitation. Further aspects and embodiments will be apparent to those of ordinary skill in the art. All documents mentioned in this specification are hereby incorporated herein by reference.

FIG. 1 amino acid sequence of the RAET1G2 polypeptide.

FIG. 2 shows the amino acid sequence of the RAET1G polypeptide

FIG. 3 shows the nucleic acid sequence encoding the full-length amino acid sequence of the RAET1G polypeptide (coding sequence (CDS) nucleotides 69 to 1072).

FIG. 4 shows the nucleic acid sequence encoding the alternatively spliced RAET1G polypeptide, consisting of aminoacid residue 1-214 of the sequence shown in FIG. 1 (coding sequence is nt 1-642).

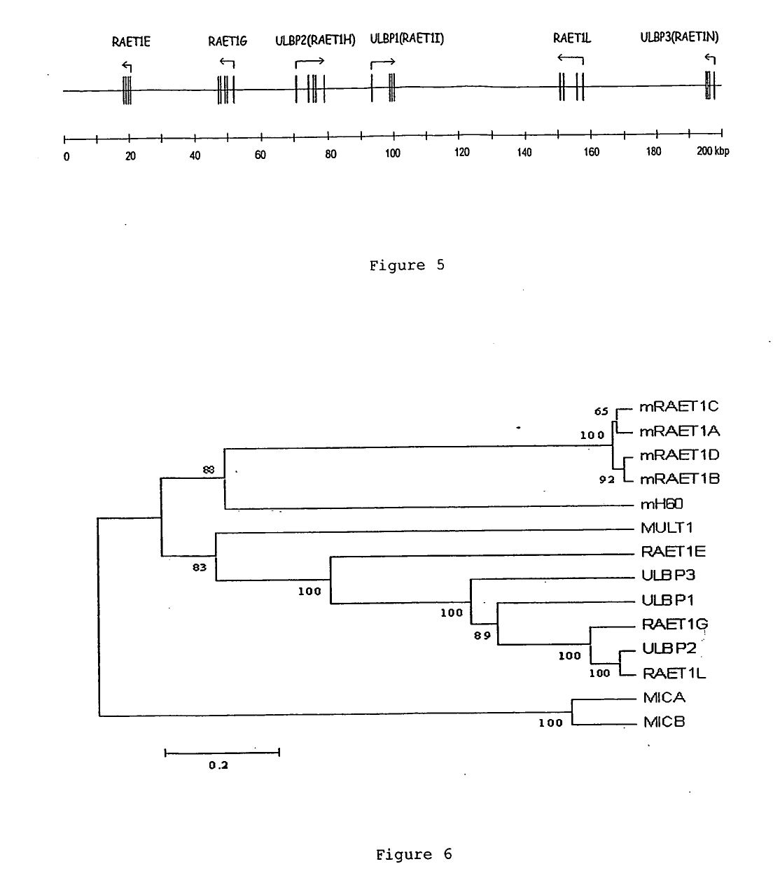

FIG. 5 shows the arrangement of expressed genes in the RAET1 cluster on chromosome 6q24.2-q25.3.

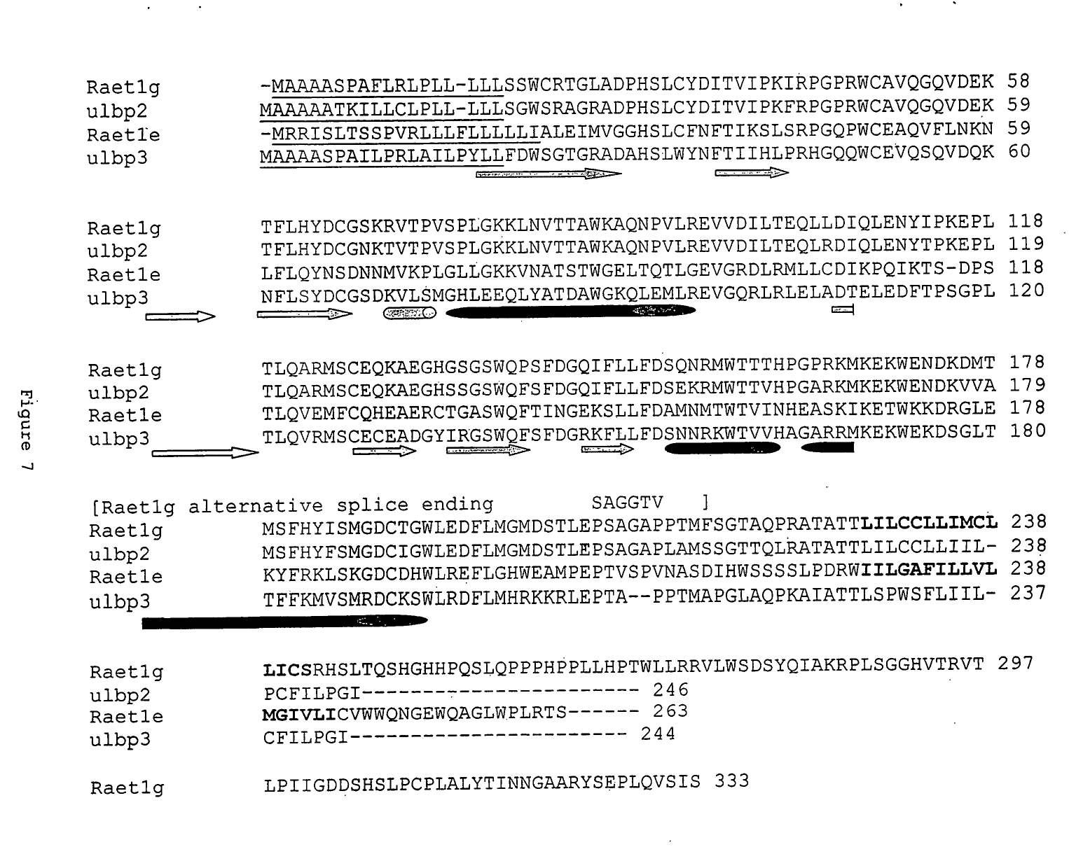

FIG. 6 shows a phylogenetic tree of murine and human NKG2D ligands. Murine ligands are identified by an ‘m’ prefix.

FIG. 7 shows a sequence alignment of RAET1G, ULBP2, RAET1E, and ULBP3. Putative TM regions are in bold letters and signal peptide sequences are underlined. Symbols indicate proposed α-helical (black cylinders), 3₁₀ helical (grey cylinder) and β-strand (grey arrow).

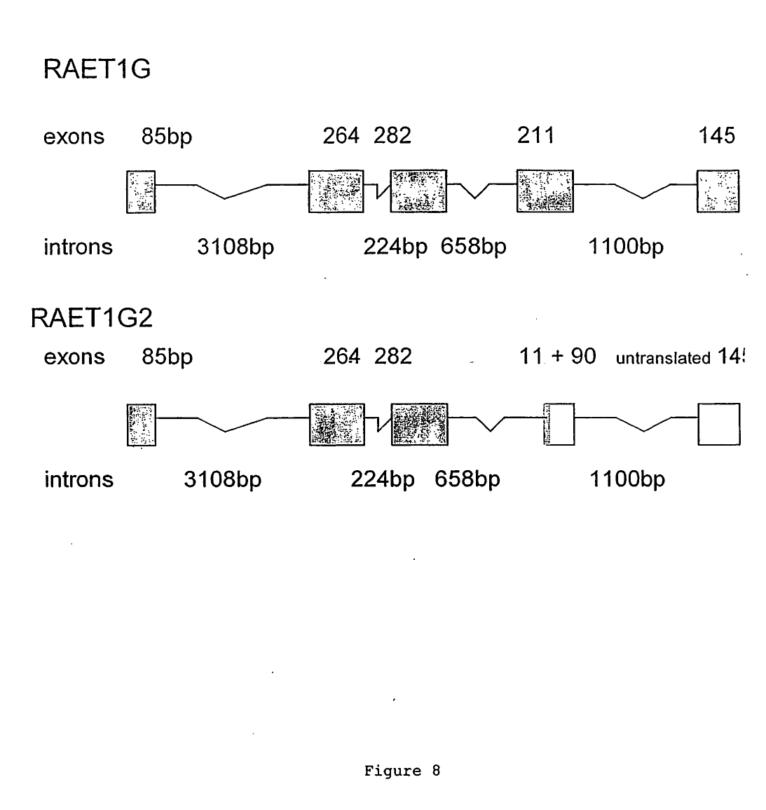

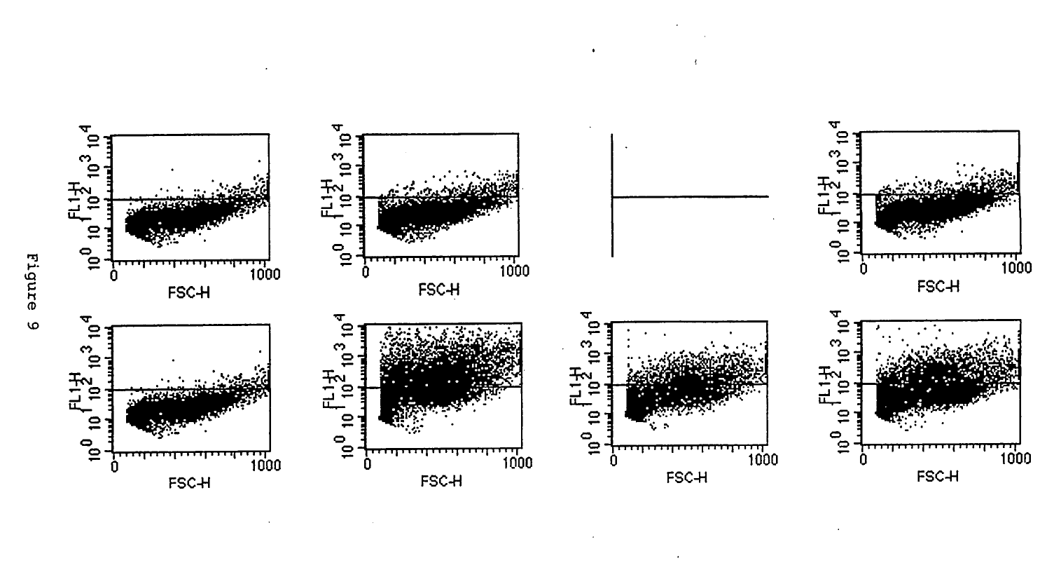

FIG. 8 shows exon structures of RAET1G and RAET1G2.

FIG. 9 shows cell surface expression of RAET1 proteins in COS-7 cells. From left to right:—mock transfected cells, ULBP2, RAET1G and RAET1E transfected cells. Upper panel stained with FITC-isotype control, Lower panel with anti-flag antibody.

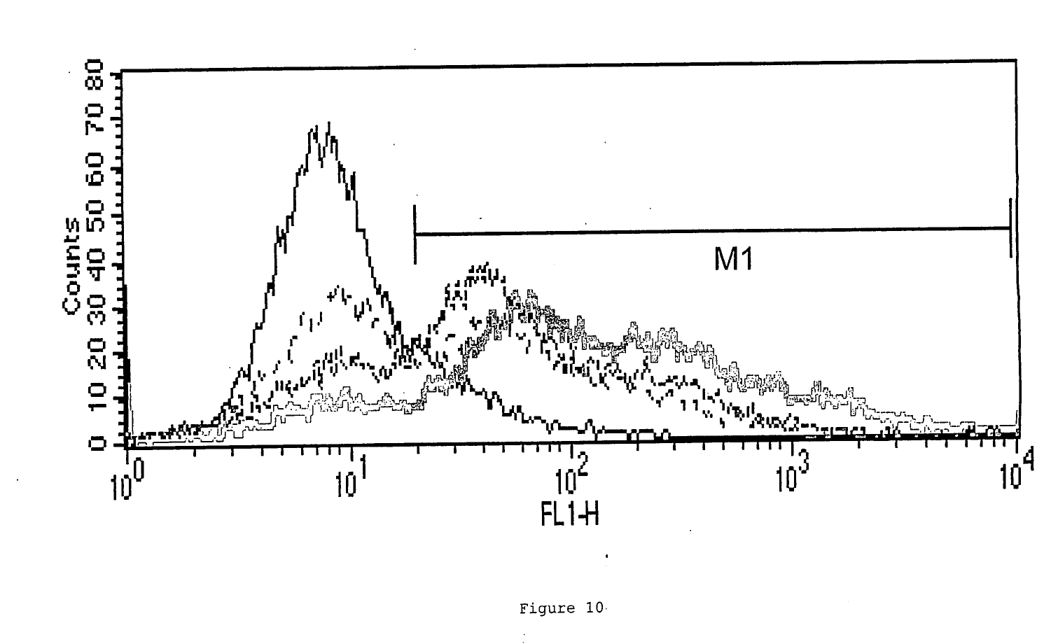

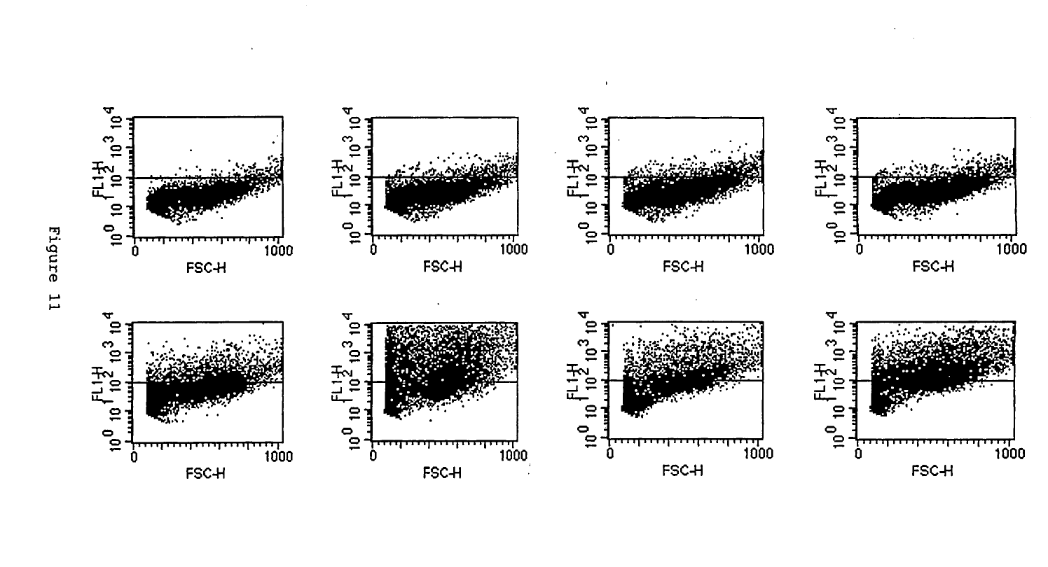

FIG. 10 shows a histogram denoting transfected cells stained with anti flag antibody—cells only (solid black line), ULBP2 (solid grey line), RAET1G (dashed grey line) and RAET1E (dashed black line).

FIG. 11 shows binding of NKG2D-Fc cells to surface-expressed RAET1 in COS-7 cells. From left to right mock transfected cells, ULBP2, RAET1G and RAET1E transfected cells. Upper panel stained with FITC-isotype control, Lower panel with NKG2D-Fc—anti human Fc-FITC.

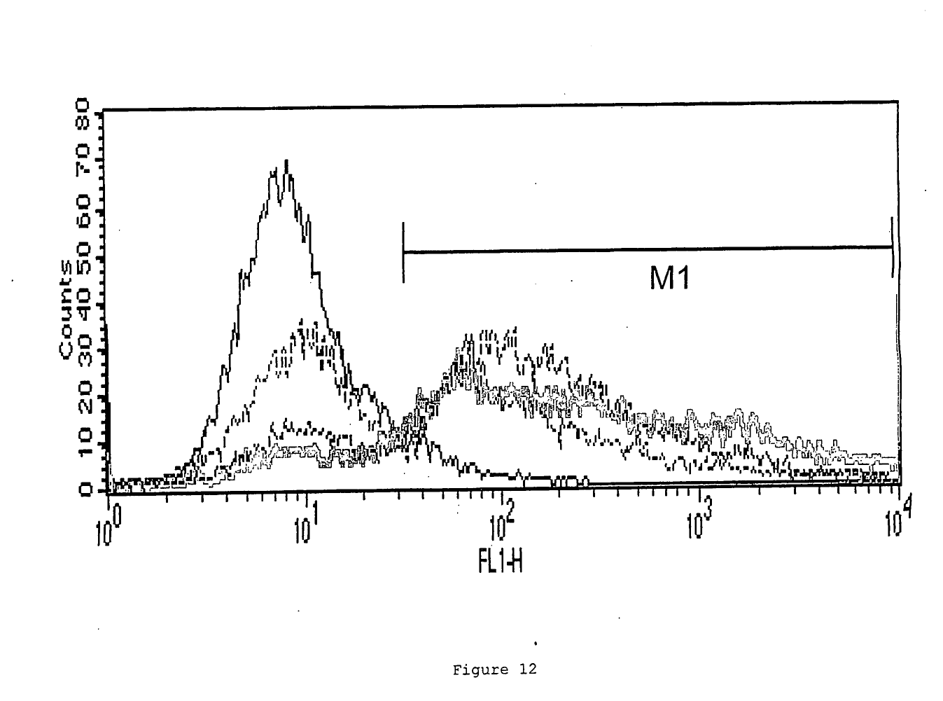

FIG. 12 shows a histogram denoting transfected cells stained with NKG2D—cells only (solid black line), ULBP2 (solid grey line), RAET1G (dashed grey line) and RAET1E (dashed black line).

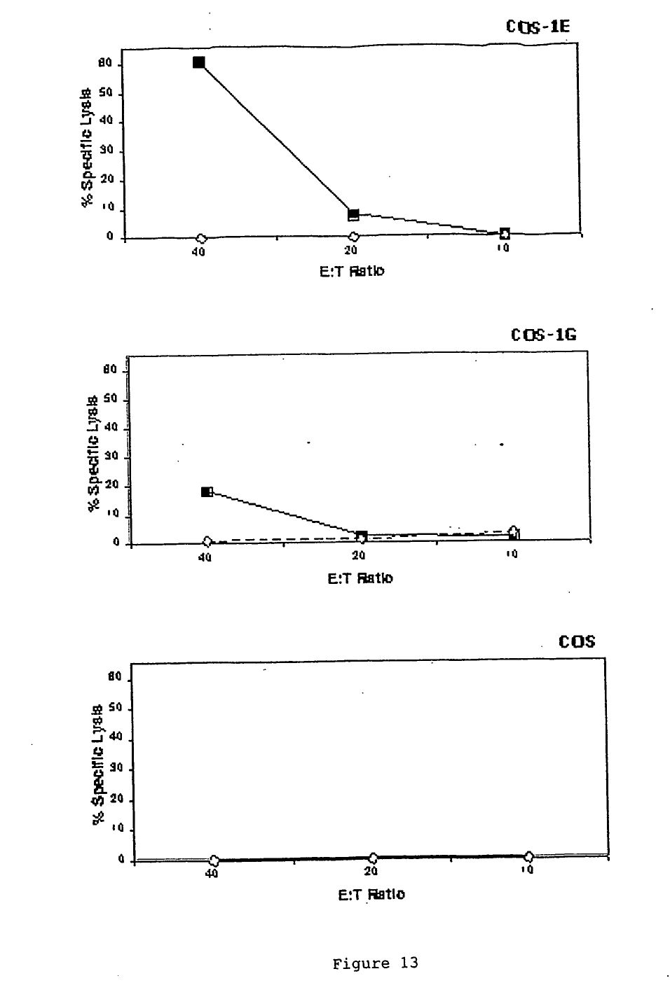

FIG. 13 shows the % specific lysis of COS-7 cells transfected with novel RAET1 molecules at different effector:target ratios. The transfected cells were incubated with either CD3⁻ CD56⁺ NKG2D⁺ human natural killer lymphocytes (black squares), or CD3⁻ CD56⁺ NKG2D⁺ human natural killer lymphocytes and NKG2D-specific monoclonal antibody (open circles). RAET1E, top panel; RAET1G, middle panel; vector only, bottom panel.

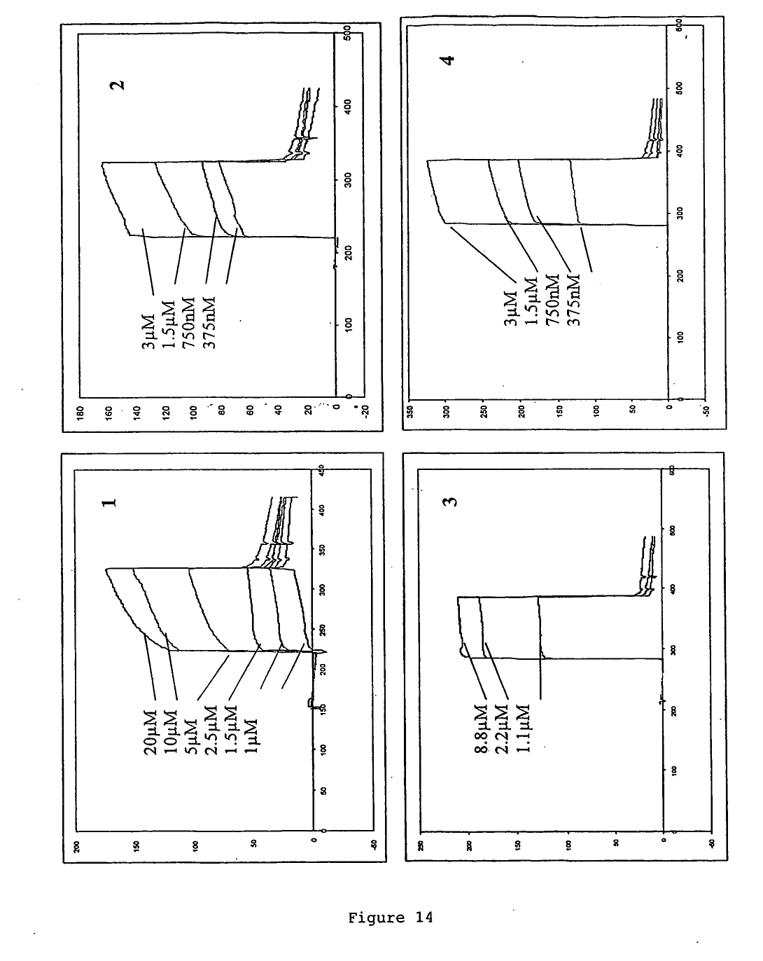

FIG. 14 shows BIACore plots used in kinetic analysis. Panels 1 and 2 show RAET1G and RAET1E binding to NKG2D-Fc respectively, at dilutions as shown. Panels 3 and 4, show RAET1G and RAET1E binding to UL16-Fc.

Table 1 shows the kinetic binding data for human RAET1/ULBP proteins binding to NKG2D and UL16.

Table 2 shows a compilation of published data for murine and human NKG2D ligands.

Table 3 shows the composition of the tissue microarrays. Number of donor samples is shown in parentheses.

Table 4 shows the distribution of RAET1G staining in tumour tissues.

### EXPERIMENTAL

Materials and Methods

Molecular Cloning of the ULBP Family

The 5′ end of RAET1G was predicted by alignment of the EST sequences AW510737, BE711112, BF513861 and the genomic DNA (contig NT_{—}023451.10). The predicted sequence matched with two IMAGE clone 3070730 and 2911855. IMAGE clone 3070730 had a truncated 3′ end and missed the stop codon. The correct 3′ end was predicted from EST AA583860 and confirmed by PCR. The signal peptide was predicted with Signal PV1.1 and the predicted transmembrane region was detected with TMpred (K. Hofmann & W. Stoffel (1993) Biol. Chem. Hoppe-Seyler 374,166). The exon structures were analysed with the GCG program (Wisconsin package) version 10.3.

The alignment was based on the global amino acid sequences or on local domains of RAET1L (NM_{—}130900.1), RAET1E (AY176317), RAET1G (AY172579), ULBP1 (NM_{—}0225218.1), ULBP2 (NM_{—}025217.2), ULBP3 (NM_{—}024518.1), MICA (BC016929), MICB (NM_{—}005922.1) and MULT1 (AK020784) and conducted using ClustalW (Higgins D et al. (1994) Nucleic Acids Res. 22:4673-4680) and PileUp. The UPGMA (unweighted pair group method with arithmetic mean) tree was constructed by the program MEGA version 2.1 (Kumar, S. et al (2001). Bioinformatics, 17, 1244-5). The consistency of the branches was assessed by bootstrap based on 1000 samples per run.

The following clones were obtained from the I.M.A.G.E clone collection, HGMP, Hinxton, Cambs, UK: RAET1G: IMAGE No. 3070730, 2911855, RAET1E: 3464637, ULBP2: 4747126 (Genbank accessions BF513861, AW 510737, BE545401, BG675590).

DNA sequencing was performed using BigDye and ABI 377 sequencer, analysis using Sequence Navigator software. Full-length receptor constructs were cloned as Flag epitope fusions in vector p3XFLAG-CMV™-9 (Sigma). Topo cloning of PCR fragments was performed using the Topo cloning kit (Invitrogen) and the manufacturer's instructions.

RT-PCR

PCR primers used for determining tissue distribution:

| | | |
|---|---|---|
| 1G | For | 5′ AGCCCCGCGTTCCTTCTA |
| | Rev | 5′ TGTATACAAGGCAAGAGGGGC |
| 1E | For | 5′ TATCCCTGACTTCTAGCCCT |
| | Rev | 5′ GCCACTCACCATTTTGCCAC |
| GAPDH | For | 5′ ACCACAGTCCATGCCATCAC |
| | Rev | 5′ TCCACCACCCTGTTGCTGTA |

Cell line RNA's were made as previously described (Allcock et al., (2003) Eur. J. Immunol. 33, 567-577). Expected sizes of products were: 935 bp for RAET1G, 835 bp for RAET1G2 and 810 bp for RAET1E. GAPDH was used as a control reaction for each cDNA.

Transfections and Flow Cytometry

Transient transfections were performed into CV-1 cells and MDCK cells using Lipofectamine 2000 (Invitrogen) and the manufacturer's standard protocol. Stably expressing cell lines of MDCK and CV1 cells were subsequently derived by selection of G418-resistant cells, by supplementing cell growth medium with 1 mg/ml G418 (Gibco).

Flow cytometry was performed on a Becton FACScalibur machine. Detection of the full-length receptors was via a FITC-conjugated monoclonal antibody to the FLAG® epitope (Sigma). Detection of NKG2D binding was via the human Fc fusion, using an anti-human IgG FITC conjugated polyclonal antibody (Dako).

NK Cell Cytotoxicity Assay

Human natural killer (NK) lymphocytes were isolated from peripheral blood using standard Ficoll isolation of mononuclear cells followed by staining with anti-CD3-FITC and anti-CD56-CyChrome (Becton Dickinson UK) monoclonal antibodies. Stained cells were analysed on a MoFlo cell sorter (Cytomation) and the CD3⁻ CD56⁺ lymphocyte population isolated. These cells were incubated in RPMI 1640 medium containing penicillin and streptomycin, 10% human AB serum and 100 U/ml recombinant interleukin-2 for three days at 37° C., 5% CO₂. Flow cytometric analysis demonstrated that this polyclonal population of NK cells were uniformly CD3⁻ CD56⁺ NKG2D⁺.

The ability of novel human RAET1 molecules to induce natural killer cell mediated lysis was assessed by an in vitro non-radioactive cytotoxicity assay (Sheehy et al., (2001) J. Immunological Methods 249, 99-110). Four wells of labelled target cells alone were set up to determine spontaneous release and each labelled target cell was assessed in duplicate at a range of effector to target ratios. For monoclonal antibody blocking, NK cell effectors were incubated at room temperature for thirty minutes in the presence of an NKG2D-specific monoclonal antibody prior to addition of labelled target cells. The percent specific lysis for each effector to target cell ratio was then calculated using % specific lysis=100−% survival.

Production of Recombinant Proteins

Soluble recombinant versions of the extracellular domain of ULBPs and RAET1E/G were produced as 6-Histidine N-terminal fusions. These were produced as inclusion bodies in E. coli BLR (DE3) using vector pT7H is derived from pGMT7 (Vales-Gomez et al., (1999) EMBO J. 18, 4250-4260) and the insoluble protein extracted with 6M Guanidine Hydrochloride. Purification and refolding in situ were performed using Ni-NTA resin (Qiagen) by sequential dilution to PBS prior to elution using PBS plus 250 mM imidazole. NKG2D-Fc fusion protein was produced from 293T cells using calcium phosphate transfection. The fusion was C-terminal to human IgG1 hinge-CH2-CH3 domains, in pcDNA3.0. UL16 Fc fusion was produced in SignalpIgplus (Sigma) as a fusion N-terminal to human IgG1 CH2-CH3. The recombinant proteins were purified using Protein A Sepharose® (Pharmacia). In all cases, eluted proteins were transferred to PBS by passing through a coarse gel filtration matrix (PD10 column, Pharmacia). SDS-PAGE and western blot analysis verified the integrity of the expressed Fc fusions. The His-tagged protein MW and purity were confirmed by SDS-PAGE, on 12% acrylamide gels based on the protocol of Laemmli. Western blotting was carried out as wet blotting transfer to Immobilon-P membrane (Millipore). Detection was using anti human-Fc HRP conjugated antibody (Dako).

Surface Plasmon Resonance (SPR)

SPR was carried out on a BIACore2000 machine. Running buffer, sensor chips and surface coupling reagents were from BIACore. Anti Human IgG (Dako) was coupled to a CM5 surface using NHS/EDC chemistry. This surface was then used for NKG2D and UL16 binding via the Fc fusion. 1.5 μg of NKG2D or UL16-Fc fusion, or a null Fc-fusion control was bound to the anti-IgG followed by sample injection at 20 μl/min. Surface regeneration was using 5 μl of 10 mM HCl. A dilution series of each ULBP was applied over NKG2D, UL16 and Fc control and the level of binding determined. Two separate batches of NKG2D, UL16 and the ULBP/RAET1 proteins were used for each determination and equivalent data obtained for each batch. Kinetic analysis was performed using BIAEvaluation 3.1 software. Separate off rates and combined global fits were performed for each NKG2D ligand dilution series.

Antibody Production

Polyclonal antibody to RAET1G was raised in rabbit using two peptides corresponding to part of the cytoplasmic domain of the protein. The peptides were:

(i) CNNGAARYSEPLQVSIS; and

(ii) CSHGHHPQSLQPPPHPP.

Peptides were manufactured and coupled to Ovalbumin by Southampton Polypeptides, (University of Southampton, UK)

The antiserum was raised using a combination of both peptides, separately coupled to ovalbumin, in rabbit by Harlan Seralabs (Place, UK). The polyclonal antibody was purified by the caprylic acid/ammonium sulphate precipitation method.

6-His tagged recombinant RAET1G was used to immunize mice in order to obtain monoclonal antibodies. Hybridoma clones were obtained from fusions of the mouse spleen after the immunization time course. These clones were initially screened by ELISA for activity. They were then screened by slot blot for ability to detect the immunogen by western blot. We have obtained good anti-serum to RAET1G from the first cloning step that gives a good response by ELISA and lights up a 50 kDa band by western blot (the expected molecular weight of RAET1G) of lysates from the cell lines K562 (erythro-leukaemia) and HT1080 (fibrosarcoma). This indicates specificity of the antibodies.

Immunohistochemistry

Immunohistochemistry was undertaken on two paraffin wax tissue microarrays [Kononen J. et al., Nat Med 1998; 4:844-847]. The first was prepared using guided tissue selection [Simon R. et al. Biotechniques 2004; 36: 98-105 transferring 2×0.6 mm diameter cores from each formalin fixed donor tissue into the recipient array. This predominantly normal tissue microarray contained a total of 342 cores from 172 donor samples as listed in Table 3. All samples were obtained from Medical Solutions plc with ethical approval obtained from the Local Research Ethics Committee. To evaluate the tissue distribution of the RAET1G antibody in tumours, sections of commercial-paraformaldehyde fixed tissue microarray (Petagen Inc, code A201(1)) containing 1 mm cores from 35 epithelial cancer samples (Table 3) were also immunostained.

To evaluate the tissue distribution of the RAET1G antibody in the small intestine, sections of commercial paraformaldehyde-fixed tissue microarray (Petagen Inc, code A201(1)) containing 1 mm cores from 10 colon and small intestine samples were also immunostained.

Automated immunohistochemistry was undertaken using a Ventana Medical Systems Discovery™ system. Sections were dewaxed, pre-treated with mild cell conditioner 1 (Tris borate/EDTA, pH8.0) then incubated in the rabbit anti RAET1G antibody at 10 μg/ml for 20 min at 37° C. For detection an avidin/biotin block preceded application of biotinylated goat anti rabbit (DakoCytomation) diluted 1/100 for 8 min at 37° C. The biotinylated antibody was then detected using a streptavidin/biotin/peroxidase kit (Ventana, DAB MAP™). The protocol was completed by automated haematoxylin counterstaining followed by manual dehydration clearing and mounting in resinous mountant.

Within each immunohistochemical run controls were included. Anti lysozyme and anti vimentin antibodies were used as positive controls to verify the antigenic preservation of the tissue cores. These controls provided positive staining in all tissue cores. To establish if any staining present in the tissues was due to non-specific interaction of the detection reagents, slides were also processed without the RAET1G antibody.

Images of the stained tissue microarray cores were automatically captured using an Ariol SL-50 automated image capture and system (Applied Imaging Inc) using a x20 objective.

Results

RAET1G has a TM Region.

Initial analysis the ULBP/RAET cluster called for six expressed genes encoding GPI linked molecules (Radosavljevic et al., (2002) Genomics 79, 114-123). We undertook detailed analysis of these sequences and identified potential TM regions in RAET1E and RAET1G. Further analysis of the genes encoding RAET1E, RAET1G and ULBP2, (RAET1E, RAET1G and RAET1H respectively) revealed a conserved exon structure, where exon 1 encoded the signal peptide and the start of the protein, exons 2 and 3 encompassed the α1 and α2 domains, and exon 4 encoded a hydrophobic sequence. In the ULBP's, this exon encoded the GPI anchor region, and 3′ UTR, but in both RAET1G and RAET1E the sequence was compatible with a TM, as well as a short cytoplasmic region (CYT). Exon 5 in RAET1G encoded the remainder of the putative cytoplasmic domain. The equivalent exon in RAET1H was silent.

To clarify the sequences of the expressed gene products, we fully sequenced clones corresponding to RAET1E and RAET1G (Radosavljevic et al. (2002) Genomics 79, 114-123). We confirmed that RAET1G was very similar to ULBP2 over the first 4 exons. A comparison of the amino acid sequence with those of existing murine and human NKG2D ligands showed that RAET1G was most closely related to ULBP2 (85% overall similarity). The highest level of amino acid (aa) identity was in the α1 and α2 domains. The remaining translated sequence encoded a TM and a 100aa CYT (FIG. 7). Similarly, analysis of the RAET1E sequence showed that it encodes two α domains then a hydrophobic TM followed by a cytoplasmic domain of 20 amino acids. RAET1E was the most divergent member of the cluster, sharing <43% identity with the other ligands, whereas ULBPs 1-3 shared 55-60% identity with each other. The conserved amino acid sequences were aligned to the key structural elements of the α1 and α2 domains by performing a ClustalW alignment using the European Bioinformatics server (EBI, Hinxton, UK), and subsequent comparison to the known crystal structure of ULBP3. The key structural features of ULBP3 are highlighted in the protein sequence alignment (FIG. 7).

Like RAET1G, the murine NKG2D ligand MULT1 also had a long CYT. No significant sequence similarity was found between the cytoplasmic regions of the two proteins. The cytoplasmic domain of RAET1G did not show homology to any proteins or domains when searched by BLAST or through Prosite. We searched for known signalling motifs in the CYT regions of RAET1E and RAET1G. No classical Immuno-Tyrosine Inhibitory Motifs (ITIM) or Immuno-Tyrosine Activating Motifs (ITAM) were identified. There was a proline-rich PxPxxP region in the cytoplasmic domain of RAET1G, which corresponded to a consensus SH3-kinase binding motif (Kay et al., (2000) FASEB J. 14, 231-241). Downstream of this were two pairs of hydrophobic residues similar to those attributed to basolateral targeting of MICA (Suemizu et al., (2002) Proc. Natl. Acad. Sci. USA 99, 2971-2976).

FIG. 6 shows a phylogenetic tree of murine and human ligands. Human and mouse NKG2D are approximately 60% identical, however their ligands are substantially different showing 25-35% identity. Therefore despite showing some similar features, such as GPI anchors or TM regions, substantial duplication and variation has occurred after speciation between mouse and man.

Alternative Splicing of RAET1G

The sequence of IMAGE clone 2911855 was colinear with RAET1G except for a 100 bp deletion, at the start of exon 4. This arrangement is compatible with alternative splicing at this boundary, with a second potential splice start shifted 3′ by 100 bp. Translation of this deleted form of RAET1G showed that the alternative splicing caused a frame-shift, and premature termination of the protein sequence. This truncated protein is predicted to be soluble, as the frame shift causes termination before the TM region. This splice form is termed RAET1G2, and its alternative sequence ending is shown below that of RAET1G in FIG. 7. Exon structures for RAET1G and RAET1G2 are shown in FIG. 8.

Expression Patterns of RAET1G/1G2 and RAET1E

Specific PCR primers were designed to establish the expression profiles of RAET1G/1G2 and RAET1E. Several tumour cell lines contained mRNA for RAET1E or RAET1G and the genes were expressed independently of each other, in cells of different lineages. This is in contrast to MICA and MICB where expression appears to be restricted to cells of epithelial origin and it is unclear whether they are expressed independently of each other (Bahram et al. (1994) Proc. Natl. Acad. Sci. 91, 6259-6263; Groh et al., (1998) Science 279, 1737-1740). The T cell leukaemia derived line HSB-2 expressed a truncated RAET1G transcript. This cDNA product was cloned using Topo cloning and, when sequenced, was identical to the splice form RAET1G2 in IMAGE clone 2911855. The expression of a splice variant encoding a soluble protein is potentially important given the proposed role of soluble NKG2D ligands in impairment of NK and T cell recognition of tumours (Groh et al., (2002) Nature 419, 734-738). A limited range of normal human tissues tested showed no expression of RAET1E, or the splice form RAET1G2. RAET1G was strongly expressed in colon, but not in other tissues screened. An EST matching RAET1G has also been identified from a larynx cDNA library.

Expression of RAET1G mRNA and Protein

Using RT-PCR on a panel of 20 normal human tissues, we detected RAET1G mRNA in only colon, consistent with restricted expression of this gene in normal human tissues.

In order to investigate the expression of RAET1G protein a polyclonal antibody was raised against peptides from its CYT. This reagent was shown to be specific by western blot analysis of cell lysates. Lysates of K562, Raji, and CV1 cells transfected with either RAET1E or RAET1G were probed. Bands corresponding to the predicted molecular weight of 50 KDa (glycosylated) were obtained for K562 and the RAET1G transfectant but not for the other cell lines. This correlates with RT-PCR data from cell lines described above. Therefore the full length RAET1G transcript is capable of being translated into a mature protein, including its unusually long CYT.

RAET1G distribution in normal tissues by immunohistochemistry RAET1G antibody binding was demonstrated in a restricted population of normal epithelial cell types. In kidney, strong punctate staining of a minority of renal tubules was observed in one of five donor samples together with uniform weaker cytoplasmic staining of several other tubules. The latter staining was non-specific as it was also observed in the control, which omitted the test antibody, and in other renal samples. Uniform cytoplasmic staining of moderate intensity was present in several follicle lining cells in all samples of thyroid whilst in colon strong punctate staining was observed in two of five samples only, indicating that RAET1G expression may vary between individuals. In the anterior pituitary, strong uniform cytoplasmic staining of the endocrine cells was observed in the four samples in which this region was fully represented. The remaining pituitary sample was composed predominantly of tissue from the pars intermedia. This tissue was unstained but scattered endocrine cells from the anterior pituitary showed intense cytoplasmic staining. In thyroid, colon and pituitary no staining was observed in the control preparation where the test antibody was omitted indicating that the staining was specific. These data confer with the mRNA distribution indicating very restricted expression of the molecule in normal physiology.

RAET1G Expression in Tumours

A more extensive distribution of epithelial staining was observed in the tumour samples (Table 4). This staining was regarded as specific as there was no evidence of equivalent staining in samples processed with omission of the test antibody. Tumours demonstrating expression of RAET1G included adenocarcinoma of colon, lung, rectum and stomach; squamous cell carcinoma of lung, oesophagus, skin and uterus, endometrioid carcinoma of uterus, follicular carcinoma of thyroid, hepatoma and cholangiocarcinoma of the liver, renal cell carcinoma, and mucinous and serous carcinoma of ovary. In some of these tumours all samples showed evidence of staining whilst in others only some of the samples were stained. The number, intensity and distribution of staining also varied across the positive samples.

The distribution of staining within the tumour samples was of particular interest. In squamous carcinoma samples a uniform cytoplasmic staining pattern was observed. This type of staining was also observed in one hepatoma sample and in serous carcinoma of the ovary. In the remaining positively stained tumours localised, predominantly punctate, cellular staining was recorded. In adenocarcinoma of colon and rectum, hepatoma (one sample), cholangoicarcinoma of the liver, mucinous carcinoma of the ovary, renal cell carcinoma, follicular carcinoma of thyroid and endometrioid carcinoma of the uterus this staining was apical or present at the margins of the tumour cells. In adenocarcinoma of the lung and stomach this staining was associated with the borders of cytoplasmic vesicles whilst in rectal adenocarinoma cellular debris or secreted protein present in the lumen of the tumour glands was also stained. In one sample of uterine squamous carcinoma only interstitial staining was observed.

RAET1G Expression in Coeliac Disease

Coeliac disease is a relatively common autoimmune condition brought on by T mediated immune responses against the patients own intestinal epithelium. NKG2D has been recently linked to villous atrophy in coeliac disease (Hue et al, 2004), with intraintestinal lymphocytes being shown to be able to kill epithelial cells via NKG2D. Using our polyclonal serum to RAET1G we investigated its expression in epithelial cells of the small intestine of normal controls in direct comparison to those of patients with coeliac disease.

Some low level punctate staining was observed in healthy small intestine samples apparently showing an intracellular distribution of RAET1G. Much stronger staining throughout the cytoplasm of the cell, and possibly at the cell surface, was observed in samples from individuals with coeliac disease. This indicates that RAET1G is upregulated in the cells of the small intestine in individuals with coeliac disease and provides indication of a role for RAET1G/NKG2D interactions in villous atrophy in patients with active coeliac disease. Therefore RAET1G may not simply be a marker of diseased tissues in celiac disease but be a direct cause of tissue damage.

RAET1E and RAET1G Expressed on the Cell Surface Bind NKG2D

Full-length cDNA's for RAET1E, RAET1G and ULBP2 were cloned as N-terminal Flag-tagged fusion proteins. These reached the cell surface in transient transfections of CV-1 cells and detection with anti-Flag antibodies in flow cytometry (FIG. 9 and FIG. 10). NKG2D, expressed as a recombinant soluble Fc fusion protein, bound to COS-7 cells transiently transfected with ULBP2, RAET1G and RAET1E by flow cytometry (FIG. 11 and FIG. 12).

RAET1E and RAET1G are Capable of Inducing NK Cell Cytotoxicity via NKG2D

RAET1E and RAET1G expressed in COS-7 cells triggered cytotoxicity by NK cells. NKG2D antibody entirely blocks this activity. Relative killing data for the two ligands and untransfected cells is shown in FIG. 13.

Binding Interactions of the ULBP Family with NKG2D and UL16

Recombinant soluble versions of ULBP1, ULBP2, ULBP3, RAET1E and RAET1G were analysed for binding to NKG2D by Surface Plasmon Resonance using a BIACore 2000 machine. Similarly, recombinant ULBP1, RAET1E and RAET1G binding was also measured to UL16. A dilution series of each ULBP protein was passed over NKG2D-Fc, UL16-Fc or Fc fusion control attached to an anti-human IgG surface. Minimal binding was seen to the Fc control surface. Examples of curves used for the kinetic global fit analyses are shown in FIG. 14, and the kinetic parameters are shown in Table 1. Table 2 shows the parameters previously determined by others for murine and human ligands (Radaev et al., (2002) J. Immunol. 169, 6279-6285; O'Callaghan et al., (2001) Immunity 15, 201-211; Carayannopoulos et al. (2002) Eur. J. Immunol. 32, 597-605; Carayannopoulos et al. (2002) J. Immunol. 169, 4079-4083).

The data presented in Table 1 were derived from at least two repeat experiments and at least two separate expressions of both Fc fusion and his-tagged proteins. Very little batch-to-batch variability was seen and all kinetic and affinity data in Table 1 have been reproduced.

The range of affinities within the complement of human and murine ligands is comparable and conforms to a similar pattern. The GPI linked murine ligands Rae1α-δ have a significantly lower affinity than the ligands with transmembrane domains, H60 and MULT1 (Table 2). Similarly, the three human GPI-anchored ULBP's have lower affinities for NKG2D than do MICA and the TM-anchored RAET1E and RAET1G molecules.

RAET1E and RAET1G had higher affinities for NKG2D than the other human NKG2D ligands, at 39 nM and 356 nM respectively. UL16 bound with varying but high affinity to the three human ligands tested, however RAET1G had a significantly higher affinity for UL16 than either RAET1E or ULBP1, and a faster on-rate, the K_{D} of 75.6 nM compared to 504 nM and 243 nM respectively.

Whilst sharing sequence similarity, the ULBP/RAET1 genes display great diversity of affinity for their shared receptor. It is striking that ULBP2 and RAET1G have 93% amino acid similarity in their α domains yet they bind NKG2D with 20 fold different affinities. One notable difference between the two is an alanine to proline substitution in RAET1G compared to ULBP2 at position 163, at the start of the helix in the α2 domains.

The presence of soluble MIC in the sera of patients with MIC⁺ tumours has been linked to a reduction in surface NKG2D on lymphocytes and may be a route for immune evasion by impairing the responsiveness of NKG2D bearing NK and T cells (Pende D. et al. (2002) Cancer Res. 62 6178-86). MICA is proposed to be lost from the cell surface of tumours through cleavage by metalloproteases (Salih et al., (2002) J. Immunol. 169, 4098-4102) and this may be the case for the TM containing ligand RAET1G. The soluble splice form of RAET1G detected in the T-cell leukaemia line HSB-2 could play a similar role.

The expression pattern of the ULBP/RAET genes presented here and in previous studies (Cosman et al., (2001) Immunity 14, 123-133; Pende et al., (2002) Cancer Res. 62, 6178-6186) shows that multiple ligands for NKG2D can be expressed on one target cell. The ligands are also clearly capable of independent expression. The data are consistent with different NKG2D ligands expressed on different tissues. MIC products are generally expressed on epithelial cells. ULBP/RAET1 can be expressed on epithelial cells but are also expressed in cell lines of non-epithelial origin, providing a rationale for roles distinct from MICA/B, for example in immune responses to lymphoid malignancies and viruses that infect lymphocytes. We show that the affinities of the human ULBP/RAET1 proteins for NKG2D are remarkably diverse, but form two groups. In line with murine data, the GPI-anchored proteins have modest to low affinities for NKG2D, whilst the ligands possessing CYT domains, such as RAET1G, have high affinity. High affinity driven by fast on-rates may be important where early signalling of infection is needed, where rapid association with the associating receptor to deal with infected cells is vital to their removal.

RAET1G may be targeted to the basolateral surface, where its fast on-rate and high affinity could make it a good front line indicator of bacterial challenge. In a polarised cell layer, such as epithelial surfaces in the gut, the differences in anchorage of NKG2D ligands allow differential distribution in the same cell, different possible signalling pathways, and hence differential availability to lymphocytes. The distribution of ligands on a cell could change on bacterial challenge, transformation, or lymphocyte engagement. The relative distribution of NKG2D ligands in distinct tissues and cellular domains may be fundamental to understanding NKG2D-mediated immune recognition.

RAET1G was shown to be a target of UL16, a molecule that is proposed to promote viral immune evasion by blocking NKG2D recognition. Both ULBP1 and RAET1G bound UL16 with higher affinity than for NKG2D, with very fast on rates. RAET1G is the highest affinity binder of UL16 with an association rate ten-fold faster for UL16 than for NKG2D. This would provide an enhanced ability to bind to viral proteins at low concentrations. The very fast on-rate and high affinity of RAET1G for UL16 indicate its potential as a pathogen recognition molecule. The unique CYT tail carried by this molecule provides the potential to transmit signals within the cell to modulate other molecules involved in responses to pathogens. This is the first NKG2D ligand described with evidence of its own signalling capability.

TABLE 1

| | | Separate k_{d} | Joint kₐ | Joint k_{d} | Derived K_{D} |
|---|---|---|---|---|---|
| Ligand | analyte | (s⁻¹) | (M⁻¹s⁻¹) | (s⁻¹) | (M) |
| NKG2D | ULBP1 | 6.9e−3 | 4.36e3 | 7.3e−3 | 1.68e−6 = |
| | | | | | 1.68 μM |
| NKG2D | ULBP2 | 3.36e−2 | 8.8e−3 | 6.3e−2 | 7.16e−6 = |
| | | | | | 7.16 μM |
| NKG2D | ULBP3 | 5e−3 | 2.35e3 | 5.37e−3 | 2.29e−6 = |
| | | | | | 2.29 μM |
| NKG2D | RAET1E | 5e−3 | 7.49e4 | 2.92e−3 | 3.9e−8 = |
| | | | | | 39 nM |
| NKG2D | RAET1G | 3e−3 | 1.05e4 | 3.74e−3 | 3.56e−7 = |
| | | | | | 356 nM |
| UL16 | ULBP1 | n/d | 2.46e3 | 5.97e−4 | 2.43e−7 = |
| | | | | | 243 nM |
| UL16 | RAET1E | n/d | 1.18e4 | 5.95e−3 | 5.04e−7 = |
| | | | | | 504 nM |
| UL16 | RAET1G | n/d | 1.15e5 | 8.71e−3 | 7.56e−8 = |
| | | | | | 75.6 nM |

TABLE 2

| | | | | |
|---|---|---|---|---|
| Analyte | kₐ (M⁻¹s⁻¹) | k_{d} (s⁻¹) | K_{D} | ref |

| | | | | | |
|---|---|---|---|---|---|
| H60 | 1.96e6 | 0.044 | 23 | nM | 27 |
| Rae1α | 4.2e5 | 0.24 | 581 | nM | 27 |
| Rae1β | 8.2e5 | 0.31 | 378 | nM | 27 |
| Rae1γ | 7.1e5 | 0.30 | 529 | nM | 27 |
| Rae1δ | 3.7e5 | 0.38 | 1.027 | μM | 27 |
| H60 | 2.1e6 | 0.03 | 14 | nM | 28 |
| MULT1 | 3.8e6 | 5.8e−3 | 2 | nM | 7 |
| ULBP3 | N/a | N/a | 4 | μM | 41 |
| MICA | 4.26e4 | 0.013 | 305 | nM | 10 |
| MICA | | | 800 | nM | 13 |

TABLE 3
Internal tissue microarray
Normal tissues:
Adrenal cortex (5), adrenal medulla (2), aorta (5), bladder (3),
breast (5), cardiac muscle (5), cerebellum (5), cerebrum (5), colon
(5), fallopian tube (5), ileum (5), kidney cortex(4), kidney medulla
(4), liver (4), lung (5), lymph node (5), oesophagus (4), ovary (3),
pancreas (5), parathyroid (1), peripheral nerve (5), pituitary (5),
placenta (5), prostate (5), skin (4), spinal cord (5), spleen (5),
stomach (3), striated muscle (5), testis (4), thyroid (5), tonsil
(5), ureter (3), uterus, endometrium (5), uterus myometrium (5).
Tumours:
Breast (4), adenocarcinoma of colon (5), kidney (4), prostate (5).
Cancer tissue microarray
Breast ductal cell carcinoma (2), colon adenocarcinoma (2), kidney
renal cell carcinoma (2), liver cholangiocarcinoma (2), liver
hepatoma (2), lung adenocarcinoma (1), lung squamous carcinoma (2),
oesophagus basaloid carcimona (1), oesophagus squamous cell
carcinoma (1), ovary mucinous carcinoma(2), ovary serous carcinoma
(2), rectal adenocarcinoma (2), skin squamous cell carcinoma (1),
stomach adenocarcinoma (1), stomach signet ring cell carcinoma (3),
thyroid gland follicular carcinoma (2), thyroid gland papilliary
carcinoma (2), uterus endometrioid carcinoma (2), uterus squamous
cell carcinoma (2).

TABLE 4

| Tissue | | Positive | |
|---|---|---|---|
| Array | Tissue Sample | samples | Staining observed |
| Internal | Colon - adenocarcinoma | 5/5 | Tumour cells, punctate apical staining of strong intensity |
| Cancer | Colon - adenocarcinoma | 1/2 | Tumour cells, punctate apical staining of moderate to strong intensity |
| | | 1/2 | Tumour cells, patchy apical weak to moderate punctate staining |
| Cancer | Kidney - renal cell carcinoma | 1/2 | Tumour cells, scattered punctate staining of moderate intensity |
| Cancer | Liver - cholangiocarcinoma | 1/2 | Punctate moderate staining of the luminal aspect scattered |
| | | | tumour cells |
| Cancer | Liver - hepatoma | 1/2 | Tumour cells, uniform moderate staining |
| | | 1/2 | Tumour cells, scattered moderate punctate staining |
| Cancer | Lung - adenocarcinoma | 1/2 | Tumour cells, scattered moderate staining of boarders of |
| | | | cytoplasmic vesicles. |
| Cancer | Lung - squamous carcinoma | 1/2 | Tumour areas show weak to strong uniform cytoplasmic staining |
| Cancer | Oesophagus - squamous cell | 1/2 | Weak patchy uniform staining of tumour |
| | carcinoma | | |
| Cancer | Ovary - mucinous carcinoma | 1/2 | Tumour cells, punctate apical staining of weak to strong intensity |
| Cancer | Ovary - serous carcinoma | 1/2 | Tumour cells, uniform weak to moderate staining |
| Cancer | Rectum - adenocarcinoma | 1/2 | Tumour cells, extensive punctate apical cytoplasmic staining of |
| | | | strong intensity supplemented by staining of shed cells in lumen. |
| Cancer | Skin - squamous cell | 1/1 | Moderate uniform staining of tumour areas |
| | carcinoma | | |
| Cancer | Stomach - adenocarcinoma | 1/1 | Tumour cells, scattered moderate staining of boarders of |
| | | | cytoplasmic vesicles. |
| Cancer | Thyroid - follicular | 1/2 | Tumour cells, patchy weak to moderate punctate staining |
| | carcinoma | 1/2 | Tumour cells, weak to strong punctate staining |
| Cancer | Uterus - endometrioid | 2/2 | Moderate staining of the margins of the tumour cells |
| | carcinoma | | |
| Cancer | Uterus - squamous carcinoma | 1/2 | Tumour areas, several cells show weak to strong uniform staining |
| | | 1/2 | Weak to moderate interstitial staining only |

1. An isolated nucleic acid which encodes a polypeptide which comprises an amino acid sequence having at least 87% sequence similarity to the amino acid sequence of FIG. 1 or FIG. 2.

2. An isolated nucleic acid according to claim 1, wherein the polypeptide comprises the amino acid sequence of FIG. 1.

3. An isolated nucleic acid according to claim 1 wherein the polypeptide comprises the amino acid sequence of FIG. 2.

4. An isolated nucleic acid according to claim 1 wherein the polypeptide binds to a UL16 and/or a NKG2D receptor.

5. An isolated nucleic acid according to claim 1 having a nucleotide sequence which has least 85% sequence identity with the nucleotide sequence of FIG. 3 or FIG. 4.

6. An isolated nucleic acid according to claim 1 wherein the isolated nucleic acid hybridises with the nucleic acid sequence shown in FIG. 3 or FIG. 4 or the complement thereof under stringent conditions.

7. An isolated polypeptide encoded by the nucleic acid according to claim 1.

8. An isolated polypeptide which is a fragment of the isolated polypeptide of claim 7 consisting of at least 110 amino acids and being able to bind to a UL16 and/or a NKG2D receptor.

9. An isolated polypeptide according to claim 7 conjugated to a functional moiety, wherein the functional moiety is a polypeptide, a non-peptidyl chemical compound, a cell or a virus particle.

10. An isolated polypeptide according to claim 9 wherein the functional moiety has cytotoxic activity or binding activity.

11. A recombinant vector comprising a nucleic acid according to claim 1.

12. A host cell comprising a heterologous nucleic acid according to claim 1.

13. A host cell according to claim 12 wherein the host cell is a bacterial cell or a eukaryotic cell.

14. A method of producing a RAET1G polypeptide comprising:
(a) causing expression from nucleic acid which encodes a RAET1G polypeptide according to claim 1 in a suitable expression system to produce the RAET1G polypeptide recombinantly; and,
(b) testing the recombinantly produced polypeptide for RAET1G activity.

15. An isolated antibody that binds specifically to a RAET1G polypeptide according to claim 7.

16. A method of identifying a disease condition in an individual, comprising: determining the presence or amount of RAET1G polypeptide in a sample obtained from the individual.

17. A method according to claim 16 wherein the condition is a cancer condition.

18. A method according to claim 16 wherein the condition is an inflammatory disease.

19. A method according to claim 18 wherein the inflammatory disease is coeliac disease.

20. A method according to claim 16 wherein the RAET1G polypeptide is soluble.

21. A method according to claim 20 wherein the soluble RAET1G polypeptide consists of amino acid sequence of FIG. 1.

22. A method according to claim 16 wherein the RAET1G polypeptide consists of the amino acid sequence of FIG. 2.

23. A method according to claim 16 wherein the presence or amount of the polypeptide is determined by contacting the sample with an antibody.

24. A method of identifying a disease condition in an individual, comprising: determining the presence or amount of a nucleic acid encoding a RAET1G polypeptide in a sample obtained from the individual.

25. A method according to claim 24 wherein the condition is a cancer condition.

26. A method according to claim 24 wherein the condition is an inflammatory disease.

27. A method according to claim 26 wherein the inflammatory disease is coeliac disease.

28. A method according to claim 24 wherein the nucleic acid encodes a soluble RAET1G polypeptide.

29. A method according to claim 28 wherein the nucleic acid comprises the nucleotide sequence of FIG. 4.

30. A method according to 24 wherein the nucleic acid comprises the nucleotide sequence of FIG. 3.

31. A method according to claim 17 wherein the sample comprises epithelial and/or epithelially derived cells.

32. A method according to claim 31 wherein the epithelial or epithelially derived cells are from the kidney, liver, lung, oesophagous, ovary, skin and/or uterus.

33. A method for obtaining and/or identifying a modulator of a RAET1G polypeptide, which method comprises:
(a) bringing into contact a RAET1G polypeptide and a test compound; and
(b) determining the interaction of the RAET1G polypeptide with the test compound.

34. A method for obtaining and/or identifying a compound which modulates the interaction of RAET1G with UL16 and/or NKG2D, which method comprises:
(a) bringing into contact a RAET1G polypeptide and a UL16 or NKG2D polypeptide in the presence of a test compound; and
(b) determining the interaction between the UL16 or NKG2D polypeptide and the RAET1G polypeptide before and after addition of the test compound.

35. A method according to claim 33 comprising identifying the test compound as a modulator of RAET1G activity.

36. A method according to claim 33 comprising isolating and/or purifying a test compound.

37. A method according to claim 33 comprising synthesising and/or manufacturing said test compound.

38. A method according to claim 33 comprising modifying the test compound to optimise the pharmaceutical properties thereof.

39. A method according to claim 33 comprising formulating the test compound in a pharmaceutical composition with a pharmaceutically acceptable excipient, vehicle or carrier.

40. A method of producing a pharmaceutical composition comprising formulating an RAET1G polypeptide according to claim 7 or fragment thereof, or nucleic acid which encodes a polypeptide which comprises an amino acid sequence having at least 87% sequence similarity to the amino acid sequence of FIG. 1 or FIG. 2 or a fragment thereof, or an antibody that binds specifically to a RAET1G polypeptide in a pharmaceutical composition with a pharmaceutically acceptable excipient, vehicle or carrier.

41. A modulator of RAET1G activity obtained by a method of claim 33.

42. A modulator of RAET1G activity according to claim 41 comprising a peptide fragment of a RAET1G polypeptide.

43. A method of treating a human or animal in need thereof for a condition mediated by RAET1G, comprising administering a RAET1G polypeptide according to claim 7 or fragment thereof, or nucleic acid which encodes a polypeptide which comprises an amino acid sequence having at least 87% sequence similarity to the amino acid sequence of FIG. 1 or FIG. 2 or a fragment thereof, an antibody that binds specifically to a RAET1G polypeptide or a modulator of a RAET1G polypeptide.

44. (canceled)

45. A method of claim 43, wherein the condition is selected from the group consisting of a pathogenic infection, a cancer condition and an immune disorder.

46.-47. (canceled)
